(19) United States
(12) Patent Application Publication
(10) Pub. No.: US 2015/0119622 A1
(43) Pub. Date: Apr. 30, 2015

(54) CATALYST FOR ALKANE OXIDATIVE DEHYDROGENATION AND/OR ALKENE OXIDATION

(71) Applicant: SHELL OIL COMPANY, Houston, TX (US)

(72) Inventors: Friso De Rooij, Amsterdam (NL); Ronald Jan Schoonebeek, Amsterdam (NL); Johanna Jacoba Berg-Slot, Amsterdam (NL); Michael Johannes Franciscus Maria Verhaak, Amsterdam (NL)

(21) Appl. No.: 14/398,508

(22) PCT Filed: May 2, 2013
(86) PCT No.: PCT/EP2013/059177

§ 371 Date: Nov. 3, 2014

(30) Foreign Application Priority Data
May 4, 2012 (EP) 12166839.6

(51) Int. Cl. B01J 27/057; C07C 5/48; B01J 23/28

(52) U.S. Cl. CPC B01J 27/0576; B01J 23/28; C07C 5/48; C07C 2523/28
USPC 585/658; 502/312; 502/215

(57) ABSTRACT

The invention relates to a process for treating a catalyst for alkane oxidative dehydrogenation and/or alkene oxidation, which catalyst is a mixed metal oxide catalyst containing molybdenum, vanadium and niobium, wherein the process comprises: contacting the catalyst with a gas mixture comprising an inert gas and oxygen (02), wherein the amount of oxygen is of from 10 to less than 10,000 parts per million by volume (ppmv), based on the total volume of the gas mixture, at an elevated temperature.

### FIELD OF THE INVENTION

The present invention relates to a process for treating a catalyst for alkane oxidative dehydrogenation (oxydehydrogenation; ODH) and/or alkene oxidation, to a process for preparing such catalyst, to the catalyst obtainable by such processes, and to an alkane ODH and/or alkene oxidation process using such catalyst.

### BACKGROUND OF THE INVENTION

It is known to oxidatively dehydrogenate alkanes, such as alkanes containing 2 to 6 carbon atoms, for example ethane or propane resulting in ethylene and propylene, respectively, in an oxidative dehydrogenation (oxydehydrogenation; ODH) process. Examples of alkane ODH processes, including catalysts and other process conditions, are for example disclosed in U.S. Pat. No. 7,091,377, WO2003064035, US20040147393, WO2010096909 and US20100256432. Mixed metal oxide catalysts containing molybdenum (Mo), vanadium (V), niobium (Nb) and optionally tellurium (Te) as the metals, can be used as such oxydehydrogenation catalysts. Such catalysts may also be used in the direct oxidation of alkenes to carboxylic acids, such as in the oxidation of alkenes containing 2 to 6 carbon atoms, for example ethylene or propylene resulting in acetic acid and acrylic acid, respectively.

It is an object of the present invention to provide a mixed metal oxide catalyst containing Mo, V, Nb and optionally Te which has a relatively high activity and/or a relatively high selectivity in the oxidative dehydrogenation of alkanes containing 2 to 6 carbon atoms, for example ethane or propane, and/or in the oxidation of alkenes containing 2 to 6 carbon atoms, for example ethylene or propylene.

### SUMMARY OF THE INVENTION

Surprisingly it was found that a mixed metal oxide catalyst containing Mo, V, Nb and optionally Te having a relatively high activity and/or a relatively high selectivity in the above-mentioned oxidative dehydrogenation process and/or above-mentioned oxidation process can be obtained by means of a process wherein the catalyst is contacted with a gas mixture comprising an inert gas and oxygen (O₂), wherein the amount of oxygen is of from 10 to less than 10,000 ppmv, at an elevated temperature.

Accordingly, the present invention relates to a process for treating a catalyst for alkane oxidative dehydrogenation and/or alkene oxidation, which catalyst is a mixed metal oxide catalyst containing molybdenum, vanadium and niobium, wherein the process comprises:

contacting the catalyst with a gas mixture comprising an inert gas and oxygen (O₂), wherein the amount of oxygen is of from 10 to less than 10,000 parts per million by volume (ppmv), based on the total volume of the gas mixture, at an elevated temperature.

Further, the present invention relates to a process for preparing a catalyst for alkane oxidative dehydrogenation and/or alkene oxidation, which catalyst is a mixed metal oxide catalyst containing molybdenum, vanadium and niobium, wherein the process comprises the above-mentioned treatment step.

Further, the present invention relates to a catalyst obtainable by any one of the above-mentioned processes.

Further, the present invention relates to a process of the oxidative dehydrogenation of an alkane containing 2 to 6 carbon atoms and/or the oxidation of an alkene containing 2 to 6 carbon atoms, wherein the catalyst obtained or obtainable by any one of the above-mentioned processes is used.

### DETAILED DESCRIPTION OF THE INVENTION

In the present invention, the catalyst is a mixed metal oxide catalyst containing molybdenum, vanadium and niobium. In addition to said three metals, the catalyst may contain other metals as well, such as for example tellurium. Preferably, the catalyst additionally contains tellurium. Thus, it is preferred that the catalyst is a mixed metal oxide catalyst containing molybdenum, vanadium, niobium and tellurium.

In the catalyst treatment process of the present invention, the catalyst which is a mixed metal oxide catalyst containing molybdenum, vanadium and niobium, is contacted with a gas mixture comprising an inert gas and oxygen (O₂), wherein the amount of oxygen is of from 10 to less than 10,000 parts per million by volume (ppmv), based on the total volume of the gas mixture, at an elevated temperature. Said catalyst treatment process may also be referred to as a catalyst calcination process. Preferably, in the present invention, such treatment is effected by subjecting the catalyst to a gas stream comprising an inert gas and oxygen (O₂), wherein the amount of oxygen is of from 10 to less than 10,000 parts per million by volume (ppmv), based on the total volume of the gas stream, at an elevated temperature.

The inert gas in said gas mixture comprising an inert gas and oxygen may be selected from the group consisting of the noble gases and nitrogen (N₂). Preferably, the inert gas is nitrogen or argon, more preferably nitrogen.

In the present invention, in said gas mixture comprising an inert gas and oxygen, the amount of oxygen is of from 10 to less than 10,000 parts per million by volume (ppmv), based on the total volume of the gas mixture. Preferably, the amount of oxygen is of from 100 to 9,500, more preferably 400 to 9,000, more preferably 600 to 8,500, more preferably 800 to 8,000, most preferably 900 to 7,500 parts per million by volume. Further, preferably, the amount of oxygen is at least 30, more preferably at least 50, more preferably at least 75, more preferably at least 100, more preferably at least 150, more preferably at least 200, more preferably at least 250, more preferably at least 300, more preferably at least 350, more preferably at least 400, more preferably at least 450, more preferably at least 500, more preferably at least 550, more preferably at least 600, more preferably at least 700, more preferably at least 800, more preferably at least 850, more preferably at least 900, most preferably at least 950 parts per million by volume. Further, preferably, the amount of oxygen is at most 9,500, more preferably at most 9,000, more preferably at most 8,500, more preferably at most 8,000, more preferably at most 7,500, more preferably at most 7,000, more preferably at most 6,500, more preferably at most 6,000, more preferably at most 5,750, more preferably at most 5,500, most preferably at most 5,250 parts per million by volume.

In the present invention, the treatment with said gas mixture comprising an inert gas and oxygen is carried out at an elevated temperature. Said elevated temperature may be of from 300 to 900° C., more preferably 400 to 800° C., more preferably 500 to 700° C., most preferably 550 to 650° C. Preferably, said temperature is at least 300° C., more preferably at least 350° C., more preferably at least 400° C., more preferably at least 450° C., more preferably at least 500° C., more preferably at least 550° C., most preferably at least 575° C. Further, preferably, said temperature is at most 900° C., more preferably at most 850° C., more preferably at most 800° C., more preferably at most 750° C., more preferably at most 700° C., more preferably at most 650° C., most preferably at most 625° C.

Further, the present invention relates to a process for preparing a catalyst for alkane oxidative dehydrogenation and/or alkene oxidation, which catalyst is a mixed metal oxide catalyst containing molybdenum, vanadium and niobium, wherein the process comprises:

a) preparing a catalyst containing molybdenum, vanadium and niobium;

b) contacting the catalyst with oxygen (O₂) at an elevated temperature, to obtain a mixed metal oxide catalyst containing molybdenum, vanadium and niobium; and

c) contacting the catalyst with a gas mixture comprising an inert gas and oxygen (O₂), wherein the amount of oxygen is of from 10 to less than 10,000 parts per million by volume (ppmv), based on the total volume of the gas mixture, at an elevated temperature.

Said catalyst preparation process comprises steps a), b) and c) which means that there may be 1 or more intermediate steps between step a) and step b) and between step b) and c) and that there may be 1 or more subsequent steps after step c). It is preferred that in the catalyst preparation process of the present invention there are no intermediate steps between step a) and step b) and between step b) and c).

The catalyst treatments in steps b) and c) of the catalyst preparation process of the present invention may also be referred to as catalyst calcinations.

Steps a) and b) of the catalyst preparation process of the present invention may be carried out in any way. Suitable procedures for carrying out those steps are disclosed in US20100256432, the disclosure of which is incorporated herein by reference.

Step a) of the catalyst preparation process of the present invention comprises preparing a catalyst containing molybdenum, vanadium, niobium and optionally tellurium. Any known way to prepare such catalyst may be applied. For example, the catalyst may be prepared by a hydrothermal process using a solution, preferably an aqueous solution, comprising molybdenum, vanadium, niobium and optionally tellurium or multiple solutions, preferably aqueous solutions, comprising one or more of said metals. Alternatively, the catalyst may be prepared by precipitation of one or more solutions, preferably aqueous solutions, comprising molybdenum, vanadium, niobium and optionally tellurium.

The latter precipitation process may comprise:

preparing two solutions, preferably aqueous solutions, one solution comprising molybdenum, vanadium and optionally tellurium, which solution is preferably prepared at slightly elevated temperature, for example 50 to 90° C., preferably 60 to 80° C., and another solution comprising niobium, which solution is preferably prepared at about, or slightly above, room temperature, for example 15 to 40° C., preferably 20 to 35° C.;

combining said two solutions resulting in a precipitate comprising molybdenum, vanadium, niobium and optionally tellurium, which said precipitate may have the appearance of a gel, slurry or dispersion;

recovering the precipitate thus obtained; and

drying the catalyst.

The precipitate thus obtained may be recovered by removing the solvent, preferably water, which can be done by drying, filtration or any other known means for recovery, preferably by drying, for example by evaporation to dryness, for example with the aid of a rotating evaporator, for example at a temperature of from 30 to 70° C., preferably 40 to 60° C., or for example by drying in an oven at a temperature of from 60 to 140° C. The recovered solid may be dried or further dried at a temperature in the range of from 60 to 150° C., suitably 80 to 130° C.

In step a) of the above-mentioned catalyst preparation process, solutions comprising molybdenum, vanadium, niobium and/or optionally tellurium, preferably aqueous solutions, may first be prepared by admixing. The elements Mo, V, Nb and optionally Te can be incorporated into the admixing step as pure metallic elements, as salts, as oxides, as hydroxides, as alkoxides, as acids, or as mixtures of two or more of the above-mentioned forms. As salts, sulfates, nitrates, oxalates, halides, or oxyhalides may be used. For example, the Mo can be incorporated as molybdic acid, ammonium heptamolybdate, molybdenum chlorides, molybdenum acetate, molybdenum ethoxide and/or molybdenum oxides, preferably ammonium heptamolybdate. The V can be incorporated as ammonium vanadate, ammonium metavanadate, vanadium oxide, vanadyl sulfate, vanadyl oxalate, vanadium chloride or vanadyl trichloride, preferably ammonium metavanadate. The Nb can be incorporated as niobium pentoxide, niobium oxalate, ammonium niobate oxalate, niobium chloride or Nb metal, preferably ammonium niobate oxalate. The optional Te can be incorporated as telluric acid, tellurium dioxide, tellurium ethoxide, tellurium chloride and metallic tellurium, preferably telluric acid.

In step b) of the catalyst preparation process of the present invention, the catalyst containing molybdenum, vanadium, niobium and optionally tellurium is contacted with oxygen at an elevated temperature, resulting in a mixed metal oxide catalyst containing molybdenum, vanadium, niobium and optionally tellurium. In the present invention, this may be effected by contacting the catalyst with a gas which substantially consists of oxygen, that is to say a gas containing more than 99.9 vol.% of oxygen, suitably 100 vol.%, at an elevated temperature. Further, this may be effected by contacting the catalyst with a gas mixture comprising an inert gas and oxygen, wherein the amount of oxygen is of from 1 to 99.9 vol.%, based on the total volume of the gas mixture, at an elevated temperature. The inert gas in said gas mixture comprising an inert gas and oxygen may be selected from the group consisting of the noble gases and nitrogen (N₂). Preferably, the inert gas is nitrogen or argon, more preferably nitrogen. In said gas mixture comprising an inert gas and oxygen, the amount of oxygen, based on the total volume of the gas, may be of from 5 to 50, more preferably 10 to 40, more preferably 15 to 30, most preferably 20 to 25 vol.%. Preferably, said gas mixture is air, which generally comprises about 78 vol.% of nitrogen and about 21 vol.% of oxygen.

Said step b) is performed at an elevated temperature, which may be in the range of from 150 to 800° C., preferably 200 to 600° C.

In step c) of the catalyst preparation process of the present invention, the catalyst is contacted with a gas mixture comprising an inert gas and oxygen (O₂), wherein the amount of oxygen is of from 10 to less than 10,000 parts per million by volume (ppmv), based on the total volume of the gas mixture, at an elevated temperature. The latter treatment is the same as the treatment in the catalyst treatment process of the present invention. Therefore, the above-described embodiments and preferences for said catalyst treatment process equally apply to this treatment step in the catalyst preparation process of the present invention.

After step c) of the catalyst preparation process of the present invention, the catalyst may be treated with a washing solution, resulting in a purified catalyst. This washing solution may comprise an acid or an oxidizer. Said acid may be an inorganic acid, such as nitric acid, or said acid may be an organic acid, such as oxalic acid. Said oxidizer may be hydrogen peroxide. Following the washing of the catalyst, the catalyst may be separated from the washing solution by filtration and the residue may be dried in air at a temperature of from 80 to 130° c.

In the present invention, the catalyst is a mixed metal oxide catalyst containing molybdenum, vanadium, niobium and optionally tellurium as the metals, which catalyst may have the following formula:

Mo₁VₐTe_{b}Nb_{c}Oₙ

wherein:

a, b, c and n represent the ratio of the molar amount of the element in question to the molar amount of molybdenum (Mo);

a (for V) is from 0.01 to 1, preferably 0.05 to 0.60, more preferably 0.10 to 0.40, more preferably 0.20 to 0.35, most preferably 0.25 to 0.30;

b (for Te) is either 0 or from >0 to 1, preferably 0.01 to 0.40, more preferably 0.05 to 0.30, more preferably 0.05 to 0.20, most preferably 0.09 to 0.15;

c (for Nb) is from >0 to 1, preferably 0.01 to 0.40, more preferably 0.05 to 0.30, more preferably 0.10 to 0.25, most preferably 0.14 to 0.20; and

n (for O) is a number which is determined by the valency and frequency of elements other than oxygen.

Further, the present invention relates to a process of the oxidative dehydrogenation of an alkane containing 2 to 6 carbon atoms and/or the oxidation of an alkene containing 2 to 6 carbon atoms, wherein the catalyst obtained by any one of the above-mentioned catalyst treatment and catalyst preparation processes or the catalyst obtainable by any one of such processes is used.

Preferably, in said alkane oxidative dehydrogenation process, the alkane containing 2 to 6 carbon atoms is a linear alkane in which case said alkane may be selected from the group consisting of ethane, propane, butane, pentane and hexane. Further, preferably, said alkane contains 2 to 4 carbon atoms and is selected from the group consisting of ethane, propane and butane. More preferably, said alkane is ethane or propane. Most preferably, said alkane is ethane.

Further, preferably, in said alkene oxidation process, the alkene containing 2 to 6 carbon atoms is a linear alkene in which case said alkene may be selected from the group consisting of ethylene, propylene, butene, pentene and hexene. Further, preferably, said alkene contains 2 to 4 carbon atoms and is selected from the group consisting of ethylene, propylene and butene. More preferably, said alkene is ethylene or propylene.

The product of said alkane oxidative dehydrogenation process may comprise the dehydrogenated equivalent of the alkane, that is to say the corresponding alkene. For example, in the case of ethane such product may comprise ethylene, in the case of propane such product may comprise propylene, and so on. Such dehydrogenated equivalent of the alkane is initially formed in said alkane oxidative dehydrogenation process. However, in said same process, said dehydrogenated equivalent may be further oxidized under the same conditions into the corresponding carboxylic acid which may or may not contain one or more unsaturated double carbon-carbon bonds. As mentioned above, it is preferred that the alkane containing 2 to 6 carbon atoms is ethane or propane. In the case of ethane, the product of said alkane oxidative dehydrogenation process may comprise ethylene and/or acetic acid, preferably ethylene. Further, in the case of propane, the product of said alkane oxidative dehydrogenation process may comprise propylene and/or acrylic acid, preferably acrylic acid.

The product of said alkene oxidation process comprises the oxidized equivalent of the alkene. Preferably, said oxidized equivalent of the alkene is the corresponding carboxylic acid. Said carboxylic acid may or may not contain one or more unsaturated double carbon-carbon bonds. As mentioned above, it is preferred that the alkene containing 2 to 6 carbon atoms is ethylene or propylene. In the case of ethylene, the product of said alkene oxidation process may comprise acetic acid. Further, in the case of propylene, the product of said alkene oxidation process may comprise acrylic acid.

The present alkane oxidative dehydrogenation process and/or alkene oxidation process may comprise subjecting a stream comprising the alkane containing 2 to 6 carbon atoms or a stream comprising the alkene containing 2 to 6 carbon atoms or a stream comprising both said alkane and said alkene to oxydehydrogenation conditions. Said stream may be contacted with an oxidizing agent, thereby resulting in oxidative dehydrogenation of the alkane and/or oxidation of the alkene. The oxidizing agent may be any source containing oxygen, such as for example air.

Ranges for the molar ratio of oxygen to the alkane and/or alkene which are suitable, are of from 0.01 to 1, more suitably 0.05 to 0.5.

Preferably, the catalyst of the present invention is used as a pelletized catalyst, for example in the form of a fixed catalyst bed, or a powdered catalyst, for example in the form of a fluidized catalyst bed.

Examples of oxydehydrogenation processes, including catalysts and other process conditions, are for example disclosed in above-mentioned U.S. Pat. No. 7,091,377, WO2003064035, US20040147393, WO2010096909 and US20100256432, the disclosures of which are herein incorporated by reference.

The amount of the catalyst in said process is not essential. Preferably, a catalytically effective amount of the catalyst is used, that is to say an amount sufficient to promote the alkane oxydehydrogenation and/or alkene oxidation reaction. Although a specific quantity of catalyst is not critical to the invention, preference may be expressed for use of the catalyst in such an amount that the gas hourly space velocity (GHSV) is of from 100 to 50,000 hr⁻¹, suitably of from 200 to 20,000 hr⁻¹, more suitably of from 300 to 15,000 hr⁻¹, most suitably of from 500 to 10,000 hr⁻¹.

In the alkane oxidative dehydrogenation process and/or alkene oxidation process of the present invention, typical reaction pressures are 0.1-20 bara, and typical reaction temperatures are 100-600° C., suitably 200-500° C.

In general, the product stream comprises water in addition to the desired product. Water may easily be separated from said product stream, for example by cooling down the product stream from the reaction temperature to a lower temperature, for example room temperature, so that the water condenses and can then be separated from the product stream.

The invention is further illustrated by the following Examples.

### Examples

### Preparation of the Catalysts

A mixed metal oxide catalyst containing molybdenum (Mo), vanadium (V), niobium (Nb) and tellurium (Te) was prepared, for which catalyst the molar ratio of said 4 metals was Mo₁V_{0.29}Nb_{0.17}Te_{0.12}. The preparation method was a precipitation method which was carried out in the following way, which was based on the Examples of US20100256432, including Example 5 of US20100256432.

Two solutions were prepared. Solution 1 was obtained by dissolving 34 g of ammonium niobate oxalate and 8.6 g of anhydrous ammonium oxalate in 340 ml of water at room temperature. Solution 2 was prepared by dissolving 76.7 g of ammonium heptamolybdate, 14.8 g of ammonium metavanadate and 12.5 g of telluric acid (Te(OH)₆) in 430 g of water at 70° C. 12.5 g of concentrated nitric acid was then added to solution 2. The 2 solutions were combined which yielded an orange gel-like precipitate. The mixture was evaporated to dryness with the aid of a rotating evaporator (“rotavap”) at 50° C.

The dried material was further dried in static air at 120° C. and then calcined in static air at 275° C. After the air calcination, the material was further calcined in a nitrogen (N₂) stream at 600° C., which stream additionally contained a different amount of oxygen (O₂) for each of the catalysts as indicated in Table 1 below. These streams containing different amounts of oxygen were provided by mixing a nitrogen stream with air in different proportions. Then the material was treated with an aqueous 5% oxalic acid solution at 80° C. and filtered and dried at 120° C. Thus, the procedures performed for preparing the catalysts only differed in terms of the amount of O₂ during the nitrogen calcination step.

### Testing of the Catalysts in Ethane Oxidative Dehydrogenation (ODH)

The catalysts thus prepared were tested for catalytic performance in ethane oxidative dehydrogenation (ODH) within a diluted small-scale testing unit under the same conditions. 500 mg of a sieve fraction of the catalyst (30-80 mesh) was loaded in a quartz reactor having an internal diameter (ID) of 4 mm. A gas stream comprising 94 vol.% of nitrogen, 4 vol.% of ethane and 2 vol.% of oxygen was passed downflow over the catalyst at a flow rate of 25 ml/minute, at atmospheric pressure and at a temperature of 350° C. The conversion of ethane and oxygen and the product composition were measured with a gas chromatograph (GC) equipped with a thermal conductivity detector (TCD). Table 1 below shows the performance of all of the differently calcined catalysts after 100 hours on stream.

TABLE 1

| | Amount O₂ during | | Ethane | Ethylene |
|---|---|---|---|---|
| Catalyst | N₂ calcination⁽¹⁾ | | conversion, % | selectivity, % |
| 1 | <10 | ppmv | 8 | n.m. |
| 2 | 500 | ppmv | 20 | 97 |
| 3 | 1,000 | ppmv | 34 | 95 |
| 4 | 2,500 | ppmv | 34 | 93 |
| 5 | 5,000 | ppmv | 32 | 93 |
| 6 | 10,000 | ppmv | <3 | n.m. |
| | (=1 | vol. %) | | |

⁽¹⁾= on the basis of the total volume of the gas stream;
n.m. = not measurable: that is to say, the conversion was too low to give a sufficiently high analytical accuracy.

1. A process for treating a catalyst for alkane oxidative dehydrogenation and/or alkene oxidation, which catalyst is a mixed metal oxide catalyst containing molybdenum, vanadium and niobium, wherein the process comprises:
contacting the catalyst with a gas mixture comprising an inert gas and oxygen (O2), wherein the amount of oxygen is of from 10 to less than 10,000 parts per million by volume (ppmv), based on the total volume of the gas mixture, at an elevated temperature.

2. A process according to claim 1, wherein the temperature is of from 300 to 900° C.

3. A process according to claim 1, wherein the amount of oxygen is of from 100 to 9,500 parts per million by volume.

4. A process according to claim 1, wherein the catalyst additionally contains tellurium.

5. A process for preparing a catalyst for alkane oxidative dehydrogenation and/or alkene oxidation, which catalyst is a mixed metal oxide catalyst containing molybdenum, vanadium and niobium, wherein the process comprises:
a) preparing a catalyst containing molybdenum, vanadium and niobium;
b) contacting the catalyst with oxygen (O2) at an elevated temperature, to obtain a mixed metal oxide catalyst containing molybdenum, vanadium and niobium; and
c) contacting the catalyst with a gas mixture comprising an inert gas and oxygen (O2), wherein the amount of oxygen is of from 10 to less than 10,000 parts per million by volume (ppmv), based on the total volume of the gas mixture, at an elevated temperature.

6. A process according to claim 5, wherein in step c) the temperature is of from 300 to 900° C.

7. A process according to claim 5, wherein in step c) the amount of oxygen is of from 100 to 9,500 parts per million by volume.

8. A process according to claim 5, wherein in step b) the temperature is of from 150 to 800° C.

9. A process according to claim 5, wherein in step b) the catalyst is contacted with air.

10. Process according to claim 5, wherein the catalyst additionally contains tellurium.

11. A catalyst obtainable by the process according to claim 1.

12. A process of the oxidative dehydrogenation of an alkane containing 2 to 6 carbon atoms and/or the oxidation of an alkene containing 2 to 6 carbon atoms, wherein the catalyst obtained by the process according to claim 1 is used.

13. A process according to claim 12, wherein the alkane is ethane or propane and the alkene is ethylene or propylene.
